(19) United States
(12) Patent Application Publication
(10) Pub. No.: US 2021/0077836 A1
(43) Pub. Date: Mar. 18, 2021

(54) WEARABLE DEVICE FOR CONTROLLING GASEOUS POLLUTANTS

(71) Applicant: POLITECNICO DI MILANO, Milano (IT)

(72) Inventors: Venere FERRARO, Milano (IT); Mila STEPANOVIC, Milano (IT)

(73) Assignee: POLITECNICO DI MILANO, Milano (IT)

(21) Appl. No.: 16/611,570

(22) PCT Filed: May 11, 2018
(86) PCT No.: PCT/EP2018/062214

§ 371 Date: Nov. 7, 2019

(30) Foreign Application Priority Data
May 23, 2017 (IT) 102017000055706

(51) Int. Cl. A62B 9/00; G08B 21/14; G08B 7/06; G01N 33/00

(52) U.S. Cl. CPC A62B 9/006; G01N 33/0063; G08B 7/06; G08B 21/14

(57) ABSTRACT

A wearable device for controlling gaseous pollutants is described; the device includes a shell container. The shell has a gaseous and vaporous pollutant sensor; and a light and vibration signal generator. The sensor is operatively connected to the generator and the generator generates said light and/or vibration signal on the basis of the signal generated by said sensor. Ina second embodiment, a method for using the device is also described. The device and method can be used for controlling gaseous and vaporous pollutants.

The present invention relates to a method and a smart device which allows harmful and/or toxic gases and vapours in insalubrious work environments, in particular in painting plants, to be detected.

### BACKGROUND OF THE INVENTION

There is an ever-present need for health prevention and protection for workers in dangerous or potentially dangerous environments and this need is regulated in different countries by specific regulations.

Among the dangerous environments we can list: Coating plants, Agriculture, Transportation, Construction sector, Body shops, Quarry, Fumigation.

Gaseous fumes, including volatile organic compounds (VOCs), carbon oxides, in particular monoxide and dioxide, and nitrogen oxides, are among the various circumstances which can put the health of the worker at risk.

Workers protect their health by means of PPE (personal protective equipment), which varies according to the working environment. In this particular case, protection of the airways is afforded by a mask.

The mask is one of the most immediate means of protection from gaseous fumes and different types of masks adapted to different levels of contamination are known.

Many industrial plants are equipped with means for controlling, containing and reducing gaseous fumes, even to such an extent that allows the worker to work safely even when not wearing the protective mask.

Nevertheless, even in the most modern plants there is a risk of malfunction in systems which control and contain the fumes, thereby exposing the worker to a potential or real danger.

There is therefore a need for a system which continually monitors the environment in which the worker works, and which warns of potential or real dangers to health, providing the information in real-time with a purpose of raising the consciousness about the exposure to health risks and therefore improve health behaviour.

The prior art has addressed this problem in various ways. For example, EP2720210 discloses a complex system for monitoring work places that are difficult to reach or see, which provides means of identifying the worker, means of capturing the image and some vital parameters of the worker, for example their breathing, and an alarm system which activates depending on the condition of the worker.

CN203898976 discloses a mask provided with a system for purifying inhaled air. The mask also comprises one or more air quality sensors which are connected to the purification system. A series of LEDs indicates the air quality.

CN102980978 discloses a system for monitoring soils polluted by VOCs, in which different levels of alarms are provided.

EP2362332 discloses general monitoring concepts and alarms in work places.

Other systems are disclosed in US2016/292988, WO2010/070360, US2016/078741, WO2016/029237 and WO2016094007.

The systems disclosed in the prior art provide either individual protection systems provided with devices for monitoring air quality, which are therefore very technically complex and also uncomfortable to wear, or centralised monitoring systems which require staff dedicated to running them.

Therefore, there is still a need for providing a device for controlling air quality which can be worn and managed by an individual worker and thus strictly personal, which constantly controls the level of gaseous and vapours pollutants, and which warns the worker in a real-time of the need to put on means of protection.

### SUMMARY OF THE INVENTION

One object of the present invention is a wearable device for monitoring air quality, in particular for volatile organic compounds (VOCs) and carbon oxides, in particular carbon dioxide, and warning of the contamination levels.

A further object of the present invention is a method for monitoring air quality, in particular for volatile organic compounds (VOCs) and carbon oxides, in particular carbon dioxide, and warning of the contamination levels in the real-time but also with a possibility of informing during the longer time periods.

These and other objects of the present invention are as defined in the independent claims, while the dependent claims refer to particular embodiments of the invention and will be described in detail with the aid of figures and examples.

### DESCRIPTION OF THE FIGURES

FIG. 1 is an illustrative exploded view of the device of the present invention.

FIG. 2 is an implementation example of the method of the present invention.

### DETAILED DESCRIPTION OF THE INVENTION

The device of the present invention, hereinafter also referred to as the “electronic nose”, is a wearable device for workers in environments exposed to gaseous contaminants, in particular volatile organic compounds (VOCs) and carbon oxides, in particular carbon dioxide.

According to the present invention, the wearable device for controlling gaseous pollutants comprises:

a. a shell container, said container comprising

b. a gaseous and vapours pollutant sensor;

c. signal generator, in particular generating a signal which attracts the attention of the user;

and is characterised in that said sensor b) is operatively connected to said generator c) and said generator c) generates said signal on the basis of the signal generated by said sensor b).

The device of the present invention can be used in environments which are subject to this type of contamination, with particular reference to coating plants.

The aim of the electronic nose is to continuously monitor the air quality, in particular VOCs and carbon dioxide, and to provide real-time feedback on the level of monitored contaminants until it generates an alarm or other type of warning on the expediency or requirement to put on suitable protection systems, for example a mask.

In one embodiment of the invention, the device communicates with a mobile device, for example a smartphone, tablet or other equivalent device which can collect and process the air quality data. The air quality data are collected long in time and provided through mobile application both as daily data and statistics about air quality. Purpose of this kind of information is long time feedback to help risk consciousness rising process. The application is collecting data from electronic nose device about the air quality in the plant during the work day and it gives comparative graph between air quality data and those collected from other embodiments of the invention (mask and breathing band). The application is used after the working activity and it has a purpose of long term information providing through graphic, about daily results and statistic based on weekly and monthly level. The interpolation of information between system's parts has a purpose of showing objectively how physiological parameters are affected due to poor protection wearing and, in this manner, educate the worker. There is a possibility of integration of another mobile application that is only for employees, that shows results about air quality in the plant based only on monitoring by the electronic nose device worn by workers. All the information collected from each part of the product's system are personal, visible and available only to workers. The benefits of implementation of mobile application next to the electronic nose device are in providing the continuous information that can be read whenever the worker wants and long in time, so that the process of consciousness about health behaviour gain is continuous.

The electronic nose of the present invention is based on sensor technology and comprises a sensor capable of detecting more substances which are specific to the type of contaminant being monitored.

In a preferred embodiment, the device of the present invention comprises a sensor for VOCs and carbon dioxide (CO₂).

The gaseous and vapours pollutant sensor is set to the maximum acceptable level of contaminants (or in other words, the minimum level of risk to health), which is determined by the various national, international or Community regulations. If the sensor detects a concentration of the contaminant above the established threshold, it generates a signal which the device of the invention processes as a warning or alarm signal clearly noticeable by the user due both to its visual language, intensity and repeatability.

The warning or alarm signal is generated by the device of the present invention as any signal which attracts the attention of the user, for example a visual or audio signal or another type of discernible signal, for example a vibration. In the device according to the present invention, said generator c) generates a visual and/or audio and/or vibration signal. The device of the invention can communicate with other devices, for example devices for collecting and processing the data on the level of VOCs or other gaseous and vapours pollutants for which the device is provided with the relevant sensor. A system for environmental monitoring is therefore also proposed, which can provide a history of the changes in the levels of gaseous and vapours pollutants, in particular VOCs, that is useful for controlling the performance of the industrial plant, in particular a painting plant, and for signalling the need for corrective intervention or planned maintenance.

Furthermore, collecting the data history allows the exposure level of the worker and the health risk to the worker to be controlled and opportunities for intervention to be provided.

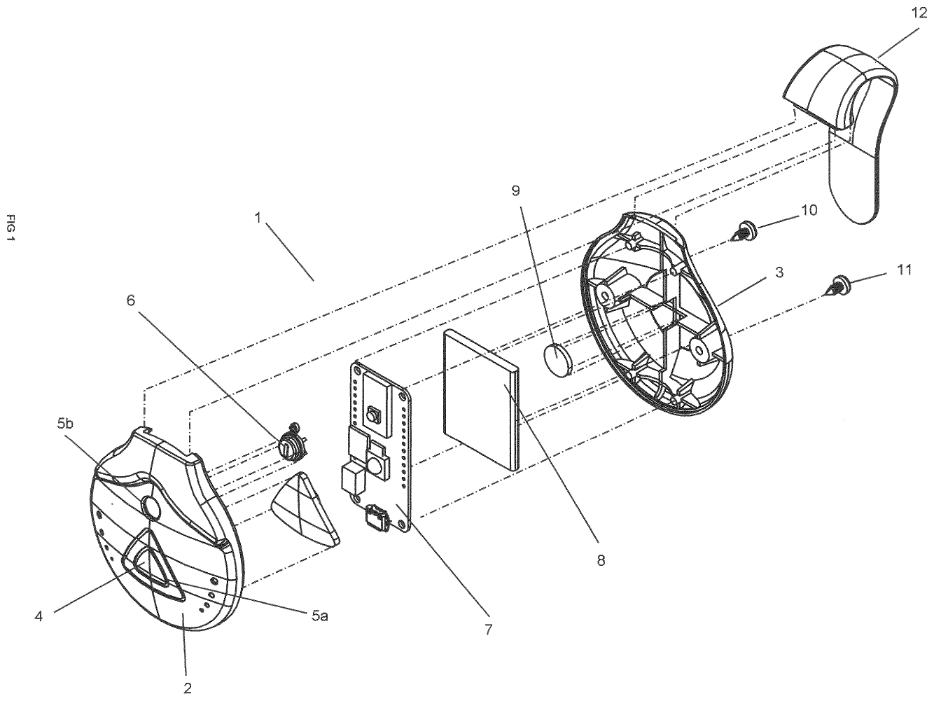

With reference to FIG. 1, one illustrative embodiment of the present invention for measuring VOCs will now be illustrated. This illustrative embodiment is intended to also be applicable to sensors for detecting other gaseous and vapours pollutants and to all other embodiments of the present invention. Therefore the invention is flexible from the point of view of both technology applicability and formal variation.

The device (1), which is shown in FIG. 1 in an exploded view, comprises an external shell, which in this example embodiment consists of two parts, a front part (2) and rear part (3) respectively.

The device (1) is advantageously provided with means for attaching onto the person who is using said device. In one embodiment, said attaching means is a clip (12), advantageously integral to the shell. Other forms of attachment are possible, for example a band, cord or belt loop.

An low engrave (4) is formed on the basically opaque surface of the front part (2) for housing a window which is coloured in tampon printing process excluding only the engraved part (5a) for viewing a device surface which emits a light signal and is contained in the shell, and for housing (5b) a button (6) for switching the device (1) on or off.

The external shell is not limited in its aesthetic shape.

The essential components b) and c) of the device are housed inside the shell.

The essential element of the device (1) is the board (7), which comprises the gaseous pollutant and vapours sensor, in particular the sensor is capable of detecting Volatile Organic Compounds (VOC) but also Tin Dioxide (stannic oxide), Solvents, Smoke, Alcohol, Carbon Monoxide and Hydrocarbons, a LED device RGB based three colour transmitter, and the power on/shutdown circuit connected to the button (6).

The device of the invention is fuelled by a lithium ion polymer rechargeable battery (8).

In a preferred embodiment, the device (1) also contains a vibration generator (9).

Constructionally, the external shell can consist of at least two separable parts, which are denoted in FIG. 1 by the reference numerals (2,3). Said parts are integrally interconnected so as to form the container for the various components of the device. The parts can be connected either by attaching means, for example screws (10,11) or joining means or both.

On the board (7), the gaseous and vapours pollutants sensor, for example the VOC sensor, is operatively connected to the light signal generator, preferably an RGB LED. According to the present invention, the light signal is generated on the basis of the signal emitted by the sensor. The variation in the light signal shows the variation in the concentration of gaseous and vapours pollutant measured by the sensor.

For example, if the light signal generator emits a single light signal, the variation in the level of pollutant detected by the sensor can be displayed by the light signal being on/off, constant or flashing. For example, if the light signal generator emits two light signals, the variation in the level of pollutant detected by the sensor can be displayed by the transition (change of colour or of intensity of the same colour) from one light signal to the other. For example, if the light signal generator emits more than two light signals, for example three, the variation in the level of pollutant detected by the sensor can be displayed by the transition (change of colour or of intensity of the same colour) from one signal to the other. For example, a first light signal, for example green, signals good air quality (no pollutant or a tolerable or admissible level of pollutant); a second light signal, for example yellow, signals a situation requiring attention or alert (level of pollutant requiring attention or level of pollutant that has exceeded the tolerable or admissible threshold, but is not yet at dangerous levels) and a third light signal, for example red, signals alarm (dangerous level of pollutant).

In a preferred embodiment of the invention, the device (1) also comprises means for generating vibrations (9), for example a motor. The means (9) is operatively connected to the sensor and the means for emitting a light signal and follows the same logic as the means for emitting a light signal. Therefore the light signal and vibrations are operating simultaneously.

Taking the situation where three signals are generated, as described above, as an example, if the signal is green, the means (9) does not vibrate; if the signal is yellow, the device (9) emits a vibration that can be of low intensity and/or frequency, but can be noticed by the person who is wearing the device, and can be continuous or intermittent; if the signal is red, the means (9) emits a vibration that is clearly perceptible to the user as being more intense than the first vibration and is preferably of a high intensity, but the frequency can be at any level, from low to high, continuous or intermittent.

The transition in both directions between the various signals can be sequential (green-yellow-red, and the corresponding vibration) or discrete (green-red).

The means for generating vibrations (9) can be adapted to any logic of the light-emitting means.

In another embodiment of the invention, the means for generating vibrations (9) can also be used when there is no means for emitting a light signal. This embodiment can be useful in situations in which the device of the invention is worn in a location that is not visible or in light conditions in which the light signal would be hard to see.

An audio signal, in the frequency audible by human ear, can be used as such or combined with any of the visual and/or vibration signal with the same logic explained above.

According to the regulations in use in the workplace, the various signals (light and/or vibration and/or visual) determine the behaviour of the worker. If there is no signal or a signal of low intensity, or, as in the example, there is a green signal, the worker is not obliged to take particular precautions against gaseous and vapours pollutants, in particular VOCs and/or carbon dioxide. If there is a signal of low intensity or, as in the example, a yellow signal, the worker is warned of a situation that could be harmful to health and is invited or obliged, according to the regulations, to take particular precautions against gaseous pollutants, in particular VOCs and/or carbon dioxide; for example, putting on a protective mask, or going to a protected location. If there is a signal of high intensity or, as in the example, a red signal, the worker is warned of a real situation that is harmful to health and is obliged, according to the regulations, to take particular precautions against gaseous and vapours pollutants, in particular VOCs and/or carbon dioxide; for example, putting on high safety PPE, for example a respirator, going to a protected location, or evacuating the workplace.

The components of the device of the present invention are known and are usually commercially available.

The sensor based on Volatile Organic Compound (VOC) detection that can be used in the present invention is any sensor among those that are known or commercially available.

In a preferred embodiment of the invention, the VOC sensor is based on MEMS technology, since this combines response speed and high accuracy with low energy consumption. This type of sensor is commercially available, for example supplied by Unitronic AG.

The means for emitting the light (visual) signal can be any one of those that are known. In a preferred embodiment of the invention, this means is an RGB LED, for example as supplied by Farnell.

The means for generating vibrations can be any one of those that are known, for example a motor as supplied by Precision Microdrives.

The means for generating visual signal can be any one of those that are known. While sensing the environment, the Electronic Nose gives three different feedbacks to the user: (i) green LED on: the sensor is operative, and the air contains an acceptable VOCs′ level; (ii) yellow LED on and low vibration: the level of VOC is starting to exceed acceptable value; (iii) red LED on and strong vibration: VOCs concentration in the air is risky for the user health.

The board that mounts the various components can be designed and manufactured together with the device of the present invention or bought preassembled on the market. In one embodiment of the invention, the board is based on the Arduino concept and can be assembled or bought preassembled. By way of example, Adafruit is a supplier of technology of this kind.

The device (1) is fuelled by a battery, for example a lithium-polymer battery.

The shell is made from any suitable material, for example plastics material, such as polypropylene, and is manufactured using common techniques, such as injection moulding.

The aim of the device according to the present invention is to protect and also educate the user through raising of health consciousness. Indeed, the warning signals and alarm, in particular the first signal (for example yellow, optionally with vibration), also make the user aware of the inherent risk in the workplace and prompt said user to take the relevant precautions, for example putting on a suitable mask, before an alarm situation arises that poses a serious risk to health. The immediate (real-time) information is effective in this case for helping to reduce the time between the realisation of the risk and safe behaviour.

The button (6), as well as switching the device (1) on and off, can be connected to the means for generating vibrations (9). The button can therefore be used to suppress the vibration. The button can also be programmed to suppress the vibration only in warning situations (for example yellow) and not in alarm situations (for example red), until the level of pollutant measured returns to safe values. The button (6) can also be independent of a further button (not shown in the figure) for switching the device (1) on or off.

In one embodiment of the invention, the device (1) is operatively connected to a device for collecting and processing the data generated by the sensor. The device for collecting and processing the data is of a known type, for example a smartphone, tablet, portable computer, or server. The device (1) can be provided with means for communicating and transmitting data, either by cable or by radio (wirelessly). For example, the device (1) is provided with a USB port, for connecting via cable, both for charging and/or data transmitting, or with a means for transmitting and receiving data wirelessly, for example Bluetooth.

In a preferred embodiment of the invention, the device for collecting and processing the data detected by the sensor is a smartphone that has a suitable application (app) installed, which provides details of the changes in the level of the gaseous and vapours pollutant, for example VOCs and/or CO₂, and/or other pollutant, in graphic and/or table form orders to help reading the information and observe comparison between this parameter and others and, for example mask wearing. The worker can thus control long in time the air quality of the work environment in which said worker works and be aware of whether said worker has taken, or has been required to take, the relevant precautions. This collecting and processing of data can be sent to a centralised monitoring system, for example a computer or a server, via the internet. This monitoring can also be used to programme or request routine medical examinations. This app is personal.

The present invention could be also used as method for detecting gaseous and vapours pollutants in work environments, in particular painting plants, that uses the device described here.

This method is useful for understanding the efficacy of the aspiration system in the coating plant. The employer can understand if the plant's aspiration system is working correctly by observing the air quality data provided in the mobile app and derived from the electronic nose device.

In addition, this kind of monitoring can be used not only during the working activity but also during the maintenance and cleaning of painting cabins in coating plant.

The method comprises:

a. switching on the device (1), whereby the signal generator located on the board (7) generates a visual signal, for example as a blue LED, showing the device is working;

b. measuring gaseous pollutants, in particular volatile organic compounds and/or carbon oxides, in particular carbon dioxide, by means of a sensor, said sensor generating a signal corresponding to the levels measured;

c. calculating the difference between said signal generated in step b) and a first reference value for the corresponding gaseous pollutant measured in step b);

d. modifying the signal generated in step a), for example as a green LED, if said difference determined in step c) is equal to zero or to a value between said first reference value and a second reference value that is greater than the first reference value showing the gaseous pollutant level is acceptable; or, alternatively or additionally e. generating a second signal, for example as a yellow LED, if said difference determined in step c) is equal to said second reference value or to a value between said second reference value and a third reference value that is greater than the second reference value showing the gaseous pollutant level has exceeded the tolerable or admissible threshold, but is not yet at dangerous levels; or, alternatively or additionally

f. generating a third signal, for example as a red LED, if said difference determined in step c) is greater than said second reference value showing the gaseous pollutant level has reached or exceeded dangerous levels.

According to an embodiment of the present invention, said signal generator generates a visual and/or audio and/or vibration signal.

According to an embodiment of the present invention, said signal is a visual and/or an audio signal.

According to an embodiment of the present invention, in step e) or f), a vibration signal is generated together with the light signal.

According to an embodiment of the present invention, in step e), said vibration signal can be suppressed.

According to an embodiment of the present invention, in step f) said vibration signal cannot be suppressed.

According to an embodiment of the present invention, step d) determines a first level of signal, or of normal operation, or of a safe environment, step e) determines a second level of signal, or of warning, or of a potentially dangerous environment, said second signal being perceived by the user as different from the first and step f) determines a third level of signal, or of alarm or of a dangerous environment, said third signal being perceived by the user as different from the second signal. According to an embodiment of the present invention, said second signal is clearly noticeable by the user and said third signal is noticeable by the user as more intense and/or noticeable than the second signal. Technical expedients can be taken in order to adapt signals to conventional rules. For example, if a visual signal is generated, it is conventional to perceive green as less intense or noticeable than yellow, which is as less intense or noticeable than red. If an audio signal is generated, it is conventional to perceive low pitch and/or frequencies as less intense or noticeable than medium/high pitch and/or frequencies. If a frequency signal is generated, it is conventional to perceive low intensities and/or frequencies as less intense or noticeable than medium/high intensities and/or frequencies.

According to an embodiment of the present invention, the volatile organic compounds (VOCs) are said principal detected pollutants.

In one embodiment of the method of the present invention, said second light signal is generated together with a vibration signal, which is intermittent or continuous and can be perceived by the user of the device.

In one embodiment of the method of the present invention, said third light signal is generated together with a vibration signal, which is intermittent or continuous and can be perceived by the user of the device, in particular as a signal that is more intense with respect to the first signal, or very intense with respect to no signal.

In one embodiment of the method of the present invention, said vibration, generated together with said second light signal, can be interrupted by means of a button located on said device.

In one embodiment of the method of the present invention, said vibration, generated together with said third light signal, cannot be interrupted by means of a button located on said device, at least until the sensor detects a level of pollutant that is zero or acceptable or admissible.

In one embodiment of the method of the present invention, the measurement of said pollutant is sent, via a data port, to a system for collecting and processing data, such as a smartphone, tablet, computer or server.

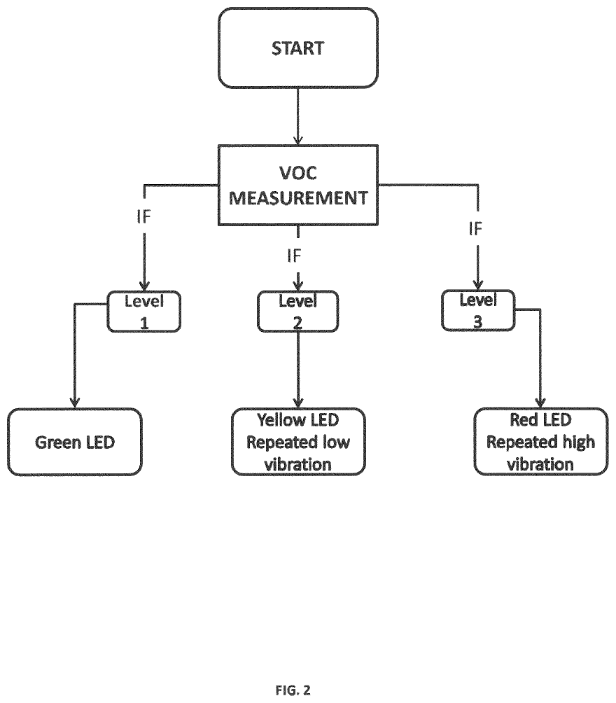

With reference to FIG. 2, an example of the method according to the present invention is provided.

When starting the device, by means of the on/off button (6), the LED emits the BLUE signal, which signals that the device is working; the VOC sensor, located on the board (7), then measures the pollutant in the ambient air. The device calculates three possible levels, based on the level of VOCs detected and compared with the stored reference values. If the difference between the measured value and the reference value is equal to zero or within an acceptable or admissible range of pollutant (level 1), the LED turns to GREEN. If, as the measurement continues, the VOC sensor detects a level of pollutant that exceeds the reference value, but still remains within an admissible range, determined by a second reference level (level 2), the LED changes the signal from GREEN to YELLOW and the vibration generator (9) starts to vibrate at a frequency and/or intensity which is/are low but can be perceived by the user. This is a signal for the user to change behaviour; said user should adopt suitable measures for personal protection, for example putting on a mask. If, as the measurement continues, the VOC sensor detects a level of pollutant that exceeds the second reference value (level 3), the LED changes the signal from YELLOW to RED and the vibration generator (9) starts to vibrate at a high frequency and/or intensity which can be perceived by the user as being more intense than the first vibration. This is a further signal for the user to change behaviour; said user should adopt further measures for personal and collective protection, for example putting on a respirator or evacuating the workplace.

The colour of the LED can also change directly from GREEN to RED, if the VOC sensor suddenly detects a level that is higher than the second reference value.

A further embodiment example of the present invention will now be described.

In a work environment in which the presence of VOCs and/or other pollutants must be controlled, for example in a paint shop, the electronic nose is worn by the user, who attaches it to their work clothing by the clip (12). The device is switched on. The blue LED indicates that the device is working normally. In normal ambient conditions, i.e. in which there are no VOCs or the level of VOCs does not pose a risk to health, the device does not generate a signal different to the green LED.

If the concentration of VOCs increases, but to levels that are still not dangerous, the green LED becomes yellow and the device emits an intermittent vibration that the user can perceive. At this point, the user, as a preventive and protective measure, puts on the protective mask and can suppress the vibration by pressing the button.

If the level of VOCs progressively or suddenly increases, the light signal changes from yellow to red, the device vibrates more intensely, and the vibration cannot be suppressed.

The user adopts further safety measures, for example putting on a respirator or evacuating the workplace.

The device of the present invention is suitable for monitoring gaseous and vapours pollutants in all situations in which this control is useful or necessary, for example in the production of paints, paint shops, car body workshops, furniture factories and artisanal enterprises where the use of paints is less regulated.

1. Wearable device for controlling gaseous pollutants, comprising:
a. a shell container, said container comprising
b. a gaseous and vaporous pollutant sensor;
c. a signal generator;
wherein said sensor b) is operatively connected to said generator c) and said signal generator c) generates a signal on the basis of a signal generated by said sensor b).

2. The device according to claim 1, wherein said sensor b) is a sensor for detecting at least one of: volatile organic compounds; carbon oxides; carbon dioxide; carbon monoxide; stannic oxide; solvents; smoke; alcohol; or hydrocarbons.

3. The device according to claim 1, wherein said signal generator c) generates the signal which attracts the attention of the user, said signal being at least one of: a visual signal an audio signal or a vibration signal.

4. The device according to claim 3, wherein said signal generator c) is a RGB LED having three colors.

5. The device according to claim 3, wherein said signal generator c) is a vibration generator.

6. The device according to claim 3, wherein said signal generator c) is a combined LED and vibration generator.

7. The device according to any claim 1, further comprising a button that is operatively connected to said signal generator c).

8. A method for detecting gaseous and vaporous pollutants in work environments that uses the device as described in claim 1, the method comprising:
a. switching on the device (1), whereby the signal generator is located on a board (7), the signal generator generates a visual signal, showing the device is working;
b. measuring gaseous pollutants, volatile organic compounds carbon oxides, or carbon dioxide, by way of a sensor, said sensor generating a signal corresponding to the levels measured;
c. calculating a difference between said signal generated in step b) and a first reference value for a corresponding gaseous pollutant measured in step b);
d. modifying the signal generated in step a) if said difference determined in step c) is equal to zero or to a value between said first reference value and a second reference value that is greater than the first reference value showing the gaseous pollutant level is acceptable; or, alternatively or additionally
e. generating a second signal if said difference determined in step c) is equal to said second reference value or to a value between said second reference value and a third reference value that is greater than the second reference value showing the gaseous pollutant level has exceeded the tolerable or admissible threshold, but is not yet at dangerous levels; or, alternatively or additionally
f. generating a third signal if said difference determined in step c) is greater than said second reference value showing the gaseous pollutant level has reached or exceeded dangerous levels.

9. The method according to claim 8, wherein said signal generator generates a signal that is at least one of: visual and/or audio or vibrational.

10. The method according to claim 9, wherein said signal is at least one of a visual or an audio signal.

11. The method according to claim 10, wherein in step e) or f), a vibration signal is generated together with the visual, audio or both visual and audio signal.

12. The method according to claim 11, wherein, in step e), said vibration signal can be suppressed.

13. The method according to claim 11, wherein, in step f) said vibration signal cannot be suppressed.

14. The method according to claim 8, wherein step d) determines a first level of signal, or of normal operation, or of a safe environment, step e) determines a second level of signal, or of warning, or of a potentially dangerous environment, said second signal being different the first signal by the user and step f) determines a third level of signal, or of alarm or of a dangerous environment, said third signal being different than the second signal by the user.

15. The method according to claim 8, wherein the volatile organic compounds (VOCs) are said pollutants.
